(19) United States
(12) Patent Application Publication
(10) Pub. No.: US 2022/0050104 A1
(43) Pub. Date: Feb. 17, 2022

(54) METHOD AND KIT FOR DETECTING T-CELLS

(71) Applicant: JACOBS UNIVERSITY BREMEN GGMBH, BREMEN (DE)

(72) Inventors: CINDY DIRSCHERL, BREMEN (DE); SEBASTIAN HARTMUT SPRINGER, BREMEN (DE)

(73) Assignee: JACOBS UNIVERSITY BREMEN GGMBH, BREMEN (DE)

(21) Appl. No.: 17/312,940

(22) PCT Filed: Nov. 19, 2019
(86) PCT No.: PCT/DE2019/200135

§ 371 Date: Jun. 11, 2021

(30) Foreign Application Priority Data
Dec. 14, 2018 (DE) 10 2018 132 210.0

(51) Int. Cl. G01N 33/569; G01N 33/543; G01N 33/566

(52) U.S. Cl. CPC G01N 33/56972; G01N 33/56977; G01N 33/566; G01N 33/543

(57) ABSTRACT

A method for detecting T cells includes a first step of bringing a plurality of different antigenic peptides into contact with peptide-free MHC molecules which are arranged in an immobilized manner on a substrate in an array so that the plurality of different antigenic peptides bind to the peptide-free MHC molecules so as to form peptide-loaded MHC molecules, a second step of bringing a sample comprising the T cells into contact with the peptide-loaded MHC molecules on the substrate, and a third step of detecting the binding of the T cells to the peptide-loaded MHC molecules on the substrate.

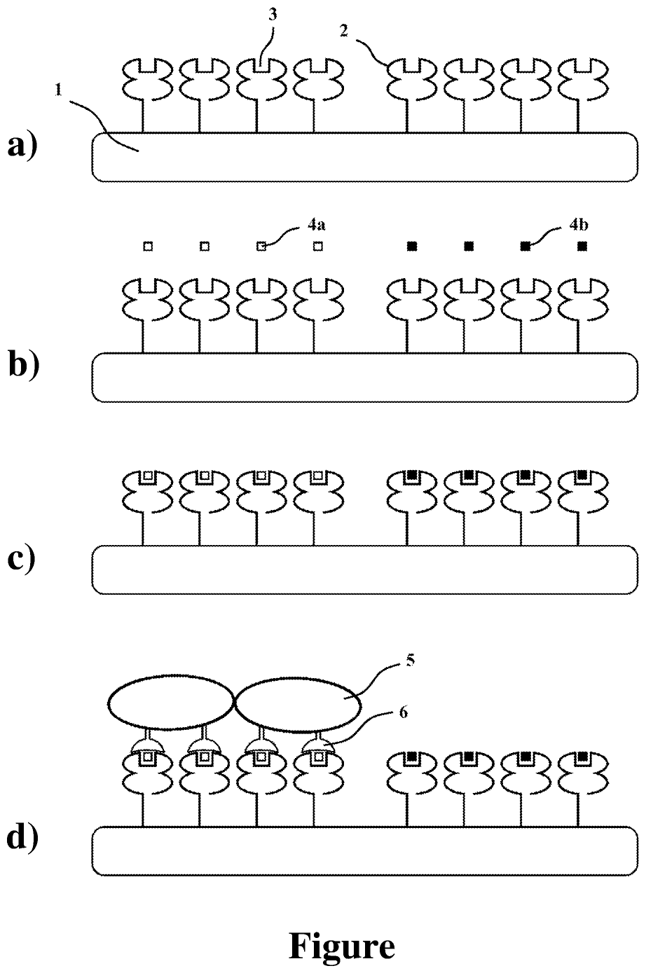

### CROSS REFERENCE TO PRIOR APPLICATIONS

This application is a U.S. National Phase application under 35 U.S.C. § 371 of International Application No. PCT/DE2019/200135, filed on Nov. 19, 2019 and which claims benefit to German Patent Application No. 10 2018 132 210.0, filed on Dec. 14, 2018. The International Application was published in German on Jun. 18, 2020 as WO 2020/119868 A1 under PCT Article 21(2).

### FIELD

The present invention relates to a method for detecting T cells and to a kit therefor.

### BACKGROUND

Personalized tumor immunotherapy is a promising approach in cancer medicine in which the aim is to activate the body's own immune system against cancer cells. T cells of the immune system generally recognize tumor cells because tumor cells show modified protein expression. Tumor-specific proteins are known as tumor antigens. Tumor antigens are not directly recognized by T cells. Tumor antigens are instead bound to a protein on the surface of the tumor cell, which is referred to as an MHC class I protein. Every type of tumor antigen bound to an MHC class I protein is recognized by a limited number of tumor-specific T cells. The T cells bind with their T cell receptor to the complex of MHC class I protein and tumor antigen.

The correct functioning of these tumor-specific T cells is often severely inhibited by the tumor. One approach for overcoming this inhibition consists in adoptive immunotherapy in which T cells are isolated from the patient, activated outside the body, and then transferred back into the patient. An adoptive cell transfer therefore requires the isolation of tumor-reactive T cells from the patient's blood. This is difficult because tumor-reactive T cells are present in the body only in very small quantities and in very low blood concentrations.

Tumor-specific T cells can be identified, for example, with the aid of their T cell receptor which binds tumor antigens that are bound to MHC class I proteins. If the antigens of the tumor are known, the tumor-reactive T cells can be identified.

Current technology consists in staining T cells with so-called MHC class I tetramers (see e.g., Bentzen A K, Hadrup S R., 2017, Evolution of MHC-based technologies used for detection of antigen-responsive T cells. Cancer Immunology, Immunotherapy 66(5):657-666. doi:10.1007/s00262-017-1971-5). These MHC class I tetramers consist of MHC class I proteins, tumor antigens, and a fluorescent dye. A significant disadvantage in the MHC tetramer staining of tumor-specific T cells is the limited quantity of blood that can be taken from a patient. Each method must therefore attempt to recognize many different T cells in a small sample. Current techniques therefore use many different tetramers at the same time to stain the T cells. Currently available methods are furthermore slow and expensive.

The article “Detection and Characterization of Cellular Immune Responses Using Peptide-MHC Microarrays” in “PLoS Biology” by Yoav Soen et al. describes a method which uses a series of peptide-MHC complexes for the rapid identification, isolation, activation, and characterization of a plurality of antigen-specific populations of T cells.

The article “Specific Capture of Peptide-Receptive Major Histocompatibility Complex Class I Molecules by Antibody Micropatterns Allows for a Novel Peptide-Binding Assay in Live Cells” in “ADVANCED SCIENCE NEWS” by Cindy Dirscherl et al. describes that antibodies pre-printed on a glass surface can be used to specifically array a peptide receptor of the immune system, i.e., the major histocompatibility complex class I molecule H-2Kb, into a defined pattern on the surface of live cells.

US 2004/0137617 A1 describes a method to capture, purify, and expand antigen-specific T lymphocytes using magnetic beads coated with recombinant MHC class I molecules.

The article “Design and use of conditional MHC class I ligands” by Mireille Toebes et al. in “Nature Medicine” describes MHC ligands that form stable complexes with MHC molecules, but which degrade on command by exposure to a defined photostimulus.

WO 2013/102458 A1 describes a method for producing an examination reagent wherein a helper ligand which enables the folding of the MHC class I protein is added to an initial solution containing a receptor protein or a receptor protein complex, more particularly an MHC class I protein or a multimeric MHC class I protein complex.

The article “Evolution of MHC-based technologies used for detection of antigen-responsive T cells” by Amalie Kai Bentzen and Sine Reker Hadrup, published in “Cancer Immunol Immunother”, provides an overview of multimer-based MHC detection technologies developed over two decades, focusing primarily on MHC class I interactions.

The article “HLA-restricted epitope identification and detection of functional T cell responses by using MHC-peptide and costimulatory microarrays” by Jennifer D. Stone et al. in “PNAS” describes a technique for screening large numbers of T cell epitopes for specific antigen recognition and functional activity induced.

The article “Dipeptides catalyze rapid peptide exchange on MHC class I molecules” by Sunil Kumar Saini, published in “PNAS”, describes dipeptides that bind to the F pocket of class I molecules.

The article “Dipeptides promote folding and peptide binding of MHC class I molecules” by Sunil Kumar Saini published in “PNAS” describes that the dipeptide glycyl-leucine supports the folding of HLA A*02:01 and H-2Kb into a peptide-receptive conformation, which rapidly binds high-affinity peptides.

### SUMMARY

An aspect of the present invention is to provide a comparatively simple, quick and cost-effective method for detecting T cells, in particular tumor-specific T cells.

In an embodiment, the present invention describes a method for detecting T cells which includes a first step of bringing a plurality of different antigenic peptides into contact with peptide-free MHC molecules which are arranged in an immobilized manner on a substrate in an array so that the plurality of different antigenic peptides bind to the peptide-free MHC molecules so as to form peptide-loaded MHC molecules, a second step of bringing a sample comprising the T cells into contact with the peptide-loaded MHC molecules on the substrate, and a third step of detecting the binding of the T cells to the peptide-loaded MHC molecules on the substrate.

### BRIEF DESCRIPTION OF THE DRAWING

The present invention is described in greater detail below on the basis of embodiments and of the drawing in which:

The FIGURE shows a simplified schematic diagram of an embodiment of the method according to the present invention.

### DETAILED DESCRIPTION

The present invention provides a method for detecting T cells, comprising the following steps:
a) bringing a plurality of different antigenic peptides into contact with peptide-free MHC molecules which are arranged in an immobilized manner on a substrate in the manner of an array so that the antigenic peptides bind to the MHC molecules, thereby forming peptide-loaded MHC molecules;
b) bringing a sample having T cells into contact with the peptide-loaded MHC molecules on the substrate; and
c) detecting the binding of T cells to the peptide-loaded MHC molecules on the substrate.

The present invention provides a method for detecting T cells using an MHC molecule array. The method according to the present invention significantly simplifies the identification of T cells, for example, tumor-specific T cells. A simplified identification of this type is advantageous in particular in situations in which identification should or must take place rapidly and automatically (e.g., in local diagnostic laboratories of a hospital).

The key concept of the present invention is the provision of an array (ordered arrangement, groups of fields), for example, an MHC molecule microarray, with “empty” MHC molecules on a suitable substrate, for example, a cell-compatible surface made of glass or tissue culture plastic. The empty MHC molecules can be rapidly loaded with specific peptides, for example, tumor-specific antigenic peptides. The empty MHC molecules can be loaded with different peptides, for example, antigens, on a substrate in a particular pattern or a particular grouped arrangement so that, for example, a first peptide is only bound to MHC molecules in a first field or region of the substrate, while a second, third, fourth etc. peptide is only bound to MHC molecules in a second, third, fourth etc. field or region of the substrate. An array of 10×10=100 different peptide/MHC molecule complexes could in this way, for example, be formed on a substrate.

T cells, for example, from a patient, can then be applied to the substrate with the loaded MHC molecules, for example, using a microfluidic instrument. The highly specific binding between the MHC molecule/peptide complex and the T cell receptors means that the T cells bind to those fields or regions of the substrate that contain the peptide/MHC molecule complexes which the T cells recognize. The T cells are therefore recruited only to some fields or regions of the substrate but not to others. The T cells on the substrate surface can be read automatically, for example, by conventional array scanners. Based on the distribution of the T cells on particular fields or regions, it is possible to measure, for example, which and how many tumor-reactive T cells are present in the patient sample.

MHC class I micropatterns with low complexity, which contain only a few different peptide/MHC class I complexes, have previously been described (see, for example, Soen Y, Chen D S, Kraft D L, Davis M M, Brown P O (2003) Detection and Characterization of Cellular Immune Responses Using Peptide-MHC Microarrays. PLOS Biology 1(3): e65. doi:10.1371/journal.pbio.0000065; Jennifer D. Stone, Walter E. Demkowicz, Lawrence J. Stern (2005), HLA-restricted epitope identification and detection of functional T cell responses by using MHC-peptide and costimulatory microarrays, Proceedings of the National Academy of Sciences March 2005, 102 (10) 3744-3749; DOI: 10.1073/pnas.0407019102). The present invention provides a high-throughput T cell array. A significant advantage of the present invention over previous T cell arrays is that the empty MHC molecule arrays can immediately be loaded with peptides for the desired screening approach and are immediately ready for use. The present invention dispenses with the need for the step of peptide exchange on an MHC class I array, for example, or the individual preparation of each MHC class I-peptide complex, which has to date been necessary for array screening approaches. This time-critical effort is significantly reduced by the array technology according to the present invention with empty MHC molecules.

An “MHC molecule” or “MHC protein” is understood to be a protein of the major histocompatibility complex, i.e., a protein that is coded by the major histocompatibility complex. MHC molecules are divided into different classes, i.e., class I and class II. The term “MHC class I protein”, “MHC class I molecule”, “class I molecule”, “MHC I molecule”, “MHC class I protein”, “class I protein” or “MHC I protein” is understood to mean a major histocompatibility complex class I protein, and the term “MHC class II protein”, “MHC class II molecule”, “class II molecule”, “MHC II molecule”, “MHC class II protein”, “class II protein” or “MHC II protein” is understood to mean a major histocompatibility complex class II protein. MHC class I and MHC class II proteins are transmembrane proteins coded by the major histocompatibility complex, MHC, and are involved in the cellular immune response. MHC class I proteins bind peptides from the cell interior, for example, from the cytosol or the lumen of the endocytic organelles, and present it to the cytotoxic T cells at the cell surface as antigen presentation. Antigens presented by MHC class II proteins cause the binding of CD-4 helper cells. An MHC I molecule generally consists of a heavy α-chain (“hc” for short, molecular weight approximately 44 kDa, approximately 350 amino acids) with three extracellular domains (α1, α2 and α3) and a transmembrane domain, and a non-covalently bonded light β-chain (also “β2-microglobulin” or “β2m” for short, approximately 12 kDa, 99 amino acids). Human MHC class I proteins are also referred to as HLA proteins or HLA antigens (HLA=human leukocyte antigen). The term includes both classic MHC class I proteins, such as HLA A, HLA B and HLA C, and non-classic MHC class I proteins, such as HLA E, HLA F and HLA G. MHC class II proteins MHC class II generally consist of an α-chain and a β-chain, each with two domains (α1, α2 and β1, β2, respectively), each chain having a transmembrane domain (α2 and β2, respectively), which anchors the MHC class II molecule on the cell membrane. The term “MHC molecule” or “MHC protein” also includes peptide-binding fragments of all these proteins, in particular the extracellular domains, as well as fusion proteins, which consist of the heavy chain (or a peptide-binding fragment of the heavy chain), possibly a linker, and the light chain. A “fragment” is in particular understood to here mean contiguous sub-sequences of at least 10, 15, 20, 25, 30, 35, 40, 45, 50, 60, 65, 70, 80, 90 or at least 100 amino acids, for example, at least 110, 120, 130, 140, 150, 160, 170, 180, 190 or at least 200 amino acids. The term also includes MHC proteins of non-human vertebrate species, for example, mouse (Mus musculus), rat (Rattus norvegicus), cattle (Bos taurus), horse (Equus equus) or rhesus macaque (Macaca mulatta). The MHC class I proteins of the mouse are, for example, referred to as H-2 proteins, which are coded by gene loci H-2K, H-2L and H-2D. A particular allotype of a mouse MHC class I protein is, for example, H-2Kb or H-2Ld.

A “peptide-free MHC molecule” or “empty MHC molecule” is understood to be an unloaded MHC molecule, i.e., an MHC molecule to which no peptide (antigen) is bound.

A peptide-loaded MHC molecule is here understood to be a complex of peptide and MHC molecule. A complex composed of peptide and MHC protein, i.e., an MHC protein, for example, an MHC class I protein, with peptide bound thereto, is also referred to here as a “peptide/MHC protein complex” or “MHC protein/peptide complex”. A complex of peptide and MHC class I protein is also referred to as a “class I peptide complex” or “peptide/MHC I”. The abbreviation “pMHC I” is also used for a complex of this type. A complex of peptide and MHC class II protein is also referred to as a “class II peptide complex” or “peptide/MHC II”. The abbreviation “pMHC II” is also used for a complex of this type. The terms “antigen-presenting MHC class I protein” or “antigen-presenting MHC class II protein” are used synonymously with the respective complexes. “Antigen-presenting” in this context means that the MHC class I protein or the MHC class II protein has a bound peptide (antigen).

The term “T cells” is understood to mean T lymphocytes. T lymphocytes carry MHC receptors (T cell receptors, TCR) in their cell membrane, with the aid of which they can recognize peptide antigens bound to MHC I proteins of other cells. The term “cytotoxic T cells” (CTL) refers to CD8+ T lymphocytes.

The term “microarray” is usually understood to mean a generally two-dimensional regular, for example, lattice- or grid-shaped, arrangement of biomolecules, for example, DNA or proteins, which is applied to a solid substrate, for example, a microscope slide, within very small, delimited areas (“spots”). These microarrays allow for the parallel analysis of a plurality of individual detections with a small quantity of biological sample material.

The peptide-free MHC molecules can be immobilized on the substrate directly or indirectly by covalent and/or non-covalent bonding. Means for directly or indirectly immobilizing proteins on a substrate are known to the person skilled in the art.

The peptide-free MHC molecules can be arranged uniformly on the substrate surface. They can, however, also be arranged on the substrate so that they are divided among individual surface portions, zones, regions or the like.

In an embodiment of the method according to the present invention, the antigenic peptides can, for example, be tumor-specific peptides, and the T cells can, for example, be tumor-specific T cells.

The antigenic peptides can, for example, be brought into contact with the peptide-free MHC molecules so that a particular pattern of peptide-loaded MHC molecules is obtained on the substrate. The peptide-free MHC molecules can, for example, be arranged so that they are distributed uniformly on the surface of the substrate, and are not, for example, restricted from the outset to particular areas, regions, zones or the like. This can take place with the aid of microfluidic printing systems. The antigenic peptides can thereby be applied on the substrate in a predefined grid. This not only allows for an automatic application of the peptides on the substrate, but also for an automatic analysis. At least step a) of bringing a plurality of different antigenic peptides into contact with peptide-free MHC molecules can, for example, take place automatically. Step c) of detecting the binding of T cells to the peptide-loaded MHC molecules on the substrate can, for example, also be performed automatically. All the above steps a), b) and c) can, for example, take place automatically.

The peptide-free MHC molecules can, for example, be loaded with different peptides so that peptide-loaded MHC molecules are arranged on the substrate within a plurality of individual fields and the MHC molecules in each field are in each case loaded with only one particular antigenic peptide, so that the MHC molecules differ from field to field by the bound antigenic peptide. The fields can, for example, be arranged in the manner of a grid or matrix to facilitate automatic processing.

The peptide-free MHC molecules can, for example, be peptide-free MHC class I molecules or MHC class II molecules, for example, peptide-free MHC class I molecules.

In the method according to the present invention, the array can, for example, be a microarray.

The detection of the bound T cells can take place using known techniques, for example, with the aid of commercial array scanners. For this purpose, the T cells can also, for example, be suitably stained, for example, using fluorescent dyes.

The present invention also relates to a kit for carrying out the method according to the present invention, the kit comprising:
a) a substrate with peptide-free MHC molecules arranged in an immobilized manner thereon in the manner of an array; and
b) a peptide library with different antigenic peptides for binding to the peptide-free MHC molecules.

A plurality of analyses can be performed simultaneously with the aid of the kit, and it is possible to, for example, determine which and how many T cells in a patient sample bind to particular tumor antigens. The peptide library can, for example, be a library of tumor-specific peptides.

The array can, for example, be a microarray.

The present invention will be explained in more detail below, purely for illustrative purposes, with the aid of the attached drawing and an application example.

The FIGURE shows an embodiment of the method according to the present invention in simplified, schematic form. The illustration is not to scale. The FIGURE shows in a) a substrate 1, for example, a glass slide, with peptide-free MHC molecules 2, here MHC class I molecules, arranged in an immobilized manner thereon. The empty binding pocket 3 of the MHC molecules 2 is illustrated here in highly simplified form as a u-shaped recess. The MHC molecules 2 are here shown arranged in two groups. In the method according to the present invention, the MHC molecules 2 can be arranged on the substrate 1 in a grouped manner from the outset as shown here. A uniform distribution on the substrate 1 is, however, preferred. Only two groups of MHC molecules 2 are here shown for the sake of clarity.

As illustrated in b) of the FIGURE, the MHC molecules 2 can be occupied in a next step with different peptides 4. Only two different peptides have here been illustrated for greater clarity, which peptides are labelled with the reference numerals 4a, 4b. In practice, however, a large number of different peptides 4 are arranged on a substrate 1, each bound by a group of MHC molecules 2. Two groups of MHC molecules 2 are illustrated in c) of the FIGURE, of which one group is occupied with a first peptide 4a and the second group with a second peptide 4b. The occupation can take place by microfluidic printing methods, wherein, as already stated above, the MHC molecules 2 can, for example, be arranged in a uniform distribution on the substrate 1 and the grid- or group-like arrangement of the peptides 4 takes place only upon occupation.

The peptide-loaded or peptide-presenting MHC molecules 2 produced in the previous step are illustrated in c) of the FIGURE.

In a next step, a sample having T cells 5, for example, tumor-specific T cells, is applied to the substrate 1 (see d) of the FIGURE). The T cells 5 bind with their T cell receptor 6 specifically to the MHC molecules 2 that present a corresponding peptide, here the MHC molecules 2 of the group illustrated on the left in the FIGURE.

### EXAMPLE—PRODUCTION OF EMPTY MHC MOLECULES

1. The gene for the heavy chain of an MHC class I molecule (examples: the human gene HLA A*02:01 and the murine gene H-2Kb) is modified according to established methods by exchanging both the codon that codes for amino acid 84 and the codon that codes for amino acid 139 with codons that code for the amino acid cysteine.
2. The gene for the heavy chain of an MHC class I molecule can be additionally modified according to established methods, for example, by attaching other amino acid sequences that facilitate purification by affinity chromatography, allow a biotinylation with the aid of the enzyme BirA, or allow the protein to adhere to surfaces, for example, glass.
3. The thus modified gene for the heavy chain of an MHC class I molecule is expressed according to established methods by expression in bacteria (for example, Escherichia coli), in insect cells (for example, Spodoptera frugiperda), or in mammal cells (for example, the cell lines HEK, 293T or CHO). The expressed protein is purified according to established methods and denatured with urea, guanidinium hydrochloride, or another chaotrope.
4. The gene for the light chain of an MHC class I molecule (also: beta-2-microglobulin) is expressed according to established methods by expression in bacteria (for example, Escherichia coli), in insect cells (for example, Spodoptera frugiperda), or in mammal cells (for example, the cell lines HEK, 293T or CHO). The expressed protein is purified according to established methods and denatured with urea, guanidinium hydrochloride or another chaotrope.
5. The heavy chain and the light chain are folded according to established methods in an in vitro folding reaction by dilution. In contrast to established methods, instead of a peptide of eight, nine, ten or more amino acids, a small molecule which supports folding is added but which itself binds only weakly, for example, an oligopeptide, in particular a dipeptide, a tripeptide, a tetrapeptide or a pentapeptide, wherein the oligopeptide can consist of proteinogenic or non-proteinogenic amino acids and is employed in concentrations in the millimolar range (see also WO 2013/102458 A1; Saini S K, Ostermeir K, Ramnarayan V R, Schuster H, Zacharias M, Springer S, 2013, Dipeptides promote folding and peptide binding of MHC class I molecules, PNAS Sep 17, 2013 110 (38) 15383-15388, doi: 10.1073/pnas.1308672110; Saini S K, Schuster H, Ramnarayan V R, Rammensee H G, Stevanović S, Springer S, 2015, Dipeptides catalyze rapid peptide exchange on MHC class I molecules PNAS Jan. 6, 2015 112 (1) 202-207; doi: 10.1073/pnas.1418690112). Small molecules are, for example, the compounds glycyl-leucine, glycyl-methionine, glycyl-cyclohexylalanine, alanyl-leucine, acetyl-isoleucine.
6. After the folding reaction, the solution is concentrated and the folded protein is separated from the by-products by established chromatographic methods. A gel filtration (size exclusion chromatography) step is also performed, which separates off the small molecule used for folding.
7. The purified protein may be biotinylated in vitro according to established methods.

The present invention is not limited to embodiments described herein; reference should be had to the appended claims.

What is claimed is:

1-8. (canceled)

9. A method for detecting T cells, the method comprising:
a first step of bringing a plurality of different antigenic peptides into contact with peptide-free MHC molecules which are arranged in an immobilized manner on a substrate in an array so that the plurality of different antigenic peptides bind to the peptide-free MHC molecules so as to form peptide-loaded MHC molecules;
a second step of bringing a sample comprising the T cells into contact with the peptide-loaded MHC molecules on the substrate; and
a third step of detecting the binding of the T cells to the peptide-loaded MHC molecules on the substrate.

10. The method as recited in claim 9, wherein,
the plurality of different antigenic peptides are tumor-specific peptides, and
the T cells are tumor-specific T cells.

11. The method as recited in claim 9, wherein the plurality of different antigenic peptides are brought into contact with the peptide-free MHC molecules so that a particular pattern of peptide-loaded MHC molecules is obtained on the substrate.

12. The method as recited in claim 11, wherein,
the peptide-loaded MHC molecules which are arranged in the immobilized manner on the substrate in the array are further arranged within a plurality of individual fields on the substrate so that each individual field of the plurality of individual fields comprises peptide-loaded MHC molecules which are bound with a particular antigenic peptide of the plurality of different antigenic peptides, and
the peptide-loaded MHC molecules differ from individual field to individual field by the particular antigenic peptide which is bound thereto.

13. The method as recited in claim 9, wherein the peptide-free MHC molecules are peptide-free MHC class I molecules or MHC class II molecules.

14. The method as recited in claim 9, wherein at least one of the first step, the second step and the third step is performed automatically.

15. The method as recited in claim 9, wherein the first step and the third step are each performed automatically.

16. The method as recited in claim 9, wherein the first step, the second step and the third step are each performed automatically.

17. The method as recited in claim 9, wherein the array is a microarray.

18. A kit for performing the method as recited in claim 9, the kit comprising:
a substrate comprising peptide-free MHC molecules which are arranged in an immobilized manner on the substrate as an array; and
a peptide library comprising different antigenic peptides for binding to the peptide-free MHC molecules.

19. The kit as recited in claim 18, wherein the array is a microarray.
